(19) United States
(12) Patent Application Publication
(10) Pub. No.: US 2016/0206742 A1
(43) Pub. Date: Jul. 21, 2016

(54) MATERIAL TO PREVENT POST SURGICAL INFECTION

(71) Applicants: Yadong Wang, Allison Park, PA (US); David S. Schwartzman, Pittsburgh, PA (US); Brian A. Klatt, Pittsburgh, PA (US)

(72) Inventors: Yadong Wang, Allison Park, PA (US); David S. Schwartzman, Pittsburgh, PA (US); Brian A. Klatt, Pittsburgh, PA (US)

(73) Assignee: University of Pittsburgh - Of the Commonwealth System of Higher Education, Pittsburgh, PA (US)

(21) Appl. No.: 14/914,611

(22) PCT Filed: Sep. 5, 2014
(86) PCT No.: PCT/US2014/054324

§ 371 Date: Feb. 25, 2016

Related U.S. Application Data
(60) Provisional application No. 61/874,735, filed on Sep. 6, 2013.

(51) Int. Cl. A61K 47/34; A61K 47/48; A61K 9/00; A61K 31/65; A61K 38/36; A61K 38/48; A61K 9/06; A61K 31/496

(52) U.S. Cl. CPC A61K 47/34; A61K 9/06; A61K 47/482; A61K 31/496; A61K 31/65; A61K 47/48215; A61K 38/363; A61K 38/4846; A61K 38/4833; A61K 9/0019; A61K 9/0085

(57) ABSTRACT

Biodegradable triblock copolymer compositions are provided which are useful in preventing and inhibiting bleeding and infection following surgery. The copolymers are reverse thermal gels in that when heated from a lower temperature to a higher temperature, they gel. These gels are useful in drug delivery when complexed with one or more, such as two or more active agents including one or more antibiotics, biofilm inhibitors, procoagulants, and/or analgesics. For example the compositions can be used for injection of active agents, such as antibiotics, procoagulants and analgesics for prevention and/or inhibition of bleeding or infection following surgery. In one example, the disclosed compositions are combined with at least one biofilm inhibitor to prevent and/or inhibit post surgical infection caused by biofilms.

### CROSS REFERENCE TO RELATED APPLICATION

The present application claims the benefit of U.S. Provisional Patent Application No. 61/874,735, entitled “Materials to Prevent Post Surgical Infection,” filed Sep. 6, 2013, the disclosure of which is incorporated herein by reference in its entirety.

### FIELD

This disclosure relates to materials that prevent bleeding and infection within wounds, such as those following a surgical procedure, and uses thereof.

### BACKGROUND

A primary concern after surgery is infection in the region of the surgical wound. The likelihood of infection is increased when there is bleeding into the region after wound closure. Infection is more likely when a foreign body is left behind, such as pacemaker, defibrillator, artificial joint, shunt, catheter, etc.

Despite best practice, post-surgical infection affects 2% to 5% of the 16 million patients undergoing surgical procedures each year in acute care hospitals. These infections can raise the risk of complications and death following surgery. Thus, modalities for decreasing the rate of infection are needed.

### SUMMARY

Disclosed herein are compositions for preventing bleeding and infection within wounds, such as those following a surgical procedure. The inventors have developed a material which, when placed into a wound region after surgery and prior to wound closure, prevents infection by the tandem effect of bleeding prevention (procoagulant) and surgical region sterilization (high dose antibiotics and/or biofilm inhibitors). The inventors have also developed a material which can prevent bleeding and pain.

In some examples, a composition comprises a thermally sensitive polymer and one or more additional active agents, such as one or more antibiotics, procoagulants, biofilm inhibitors, analgesics or a combination thereof. The disclosed compositions are thermally sensitive in physical properties (e.g., may transition from liquid at room temperature to gel at body temperature), biodegradable, and elute active agents, such as asynchronously elute 2 or more types of agents, including antibiotics, biofilm inhibitors, and/or procoagulants. The compositions can be adjusted as to the type, number, and release kinetics of the enclosed substances as desired by one of skill in the art. In some examples, a single composition comprises the one or more antibiotics, biofilm inhibitors, or procoagulants. In other examples, a single composition comprises one or more antibiotics and/or biofilm inhibitors and a second composition comprises one or more procoagulants. In further examples, a single composition comprises one or more procoagulants and one or more analgesics. Thus, the number of active agents in a single composition may vary as desired.

In some examples, a composition comprises is a reverse thermal gel composition comprising a triblock copolymer having the structure B-A-B in which A is one of a polyurethane or poly(ester urethane) group that comprises one or more pendant active groups, blocked active groups or active agents and B is a hydrophilic block that can be PEG of various sizes, hyaluronan of various sizes, poly(vinyl alcohol) or oligo(vinyl alcohol), polycarbohydrage, etc. and one or more additional active agents, such as one, two, three, four, five, etc. or more antibiotics, biofilm inhibitors, procoagulants, analgesics or a combination thereof. The composition is in solution at a lower temperature, e.g., at room temperature and transitions to a gel as the temperature is raised, to form a complete gel at a higher temperature, e.g., physiological (body) temperature (e.g., 35° C.-40° C.). The triblock copolymer may be converted to a pharmaceutically acceptable salt.

In some examples, the one or more, such as two or more active agents comprise an antibiotic, biofilm inhibitor, a procoagulant, an analgesic, or any combination thereof. In some examples, a disclosed composition includes a disclosed reverse thermal gel composition and one or more antibiotics either alone or in combination with a procoagulant and/or analgesic (pain medication). In some examples, a disclosed composition includes a disclosed reverse thermal gel composition and one or more biofilm inhibitors. In some examples, a disclosed composition includes a disclosed reverse thermal gel composition and one or more biofilm inhibitors with one or more antibiotics, procoagulants and/or analgesic. In some examples, the one or more antibiotic agents is one or more antimycobacterial agents and/or one or more broad-spectrum tetracycline antibiotic agents. In some examples, the one or more antimycobacterial agents is rifampicin, rifaximin, dapsone, ampicillin, norfloxacin, silver sulfadiazine, tigecycline, cefoperazone, sulfisoxazole, hydrocortisone/acetic acid, gemifloxacin, rifampin/isoniazid, rifampin/isoniazid/pyrazinamide

In some examples, the antimycobacterial agent is rifampicin.

In some examples, the one or more antibiotics is one or more broad-spectrum tetracycline antibiotics. In some examples, the broad-spectrum tetracycline antibiotic is minocycline and/or doxycycline. In some examples, the broad-spectrum tetracycline antibiotic is minocycline. These examples are not representative of an exhaustive list, as other antibiotics may be adapted to this approach,

In some examples, the composition comprises at least one procoagulant agent. In some examples, the one or more procoagulants is fibrinogen, prothrombin, factor Xa, and thrombin. These examples are not representative of an exhaustive list, as other procoagualants may be adapted to this approach.

In some examples, the composition comprises one or more biofilm inhibitors, such as at least one, two or more biofilm inhibitors.

Methods of preventing and/or inhibiting wound bleeding and/or infection are also disclosed herein. In some examples, methods of preventing and/or inhibiting wound bleeding and/or infection following surgery are disclosed comprising administering a disclosed composition to a subject following a surgical procedure. In some examples, methods of preventing and/or inhibiting wound bleeding and/or infection in wounds which by design enclose a foreign body, such as pacemaker implantation pockets, joint replacement sites or neurosurgical sites are disclosed. In some examples, methods of preventing and/or inhibiting wound bleeding and/or infection in wounds which do not include a foreign body are disclosed.

The disclosed compositions can be delivered to the site of interest by methods known to those of skill in the art. In some examples, the disclosed compositions are delivered by injection, spray, coating, embedding and other like modes of delivery. The active agents within the polymer composition may vary as depending upon the type of wound and/or infection, the desired duration of treatment or the combination thereof. In some examples, a disclosed composition comprising one or more antibiotics, procoagulant, and analgesic is administered, such as by injection, spray, coating, embedding and other like modes of delivery, to a wound following surgery, such as to a primary cartilaginous joint to prevent or inhibit bleeding or infection. In one particular example, a disclosed composition comprising one or more, such as two or more antibiotics, biofilm inhibitors, procoagulant, and analgesic is administered, such as by injection, spray, coating, embedding and other like modes of delivery, to a knee following surgery to prevent or inhibit bleeding or infection.

In some examples, a disclosed composition comprising one or more procoagulants is administered, such as by injection, following primary total joint arthroplasty (TJA). In some examples, a disclosed composition comprising one or more antibiotics, biofilm inhibitors, procoagulant, and analgesic is administered, such as by injection, spray, coating, embedding and other like modes of delivery, to a subject who is at risk of infection or excessive bleeding, such as a subject who has undergone surgery.

The foregoing and other features and advantages of the disclosure will become more apparent from the following detailed description, which proceeds with reference to the accompanying figures.

### BRIEF DESCRIPTION OF THE DRAWINGS

FIG. 1 is a digital image showing a pacemaker in a closed pocket.

FIG. 2 is a digital image showing a pocket without a disclosed reverse thermal gel composition.

FIG. 3 is a digital image showing a pocket including an exemplary reverse thermal gel composition.

FIG. 4 is a graph illustrating cumulative release of minocycline HCl and rifampicin.

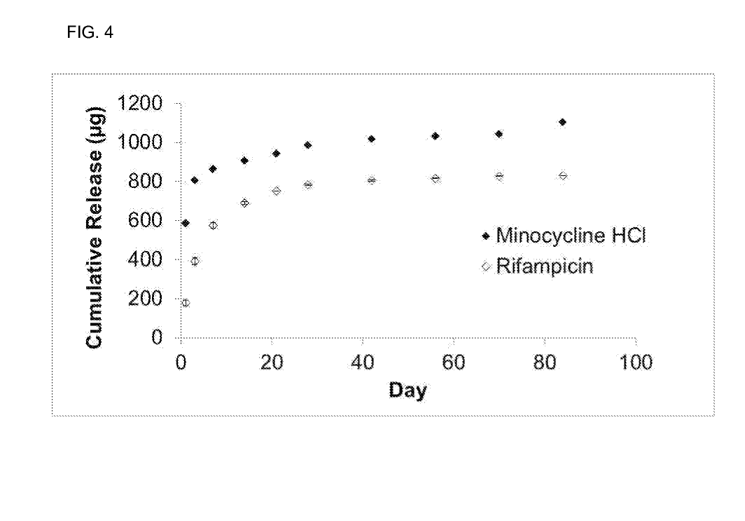

### DETAILED DESCRIPTION

This application is related to U.S. patent application Ser. No. 13/581,518 which is related to PCT Application No. PCT/US11/27233 which claims the benefit of U.S. Provisional Application Nos. 61/310,874, filed Mar. 5, 2010, 61/389,491, filed Oct. 4, 2010, and 61/426,514, filed Dec. 23, 2010, each of which is incorporated herein by reference in its entirety.

### I. Terms

As used herein, the term “polymer composition” is a composition comprising one or more polymers. As a class, “polymers” includes homopolymers, heteropolymers, co-polymers, block polymers, block co-polymers and can be both natural and synthetic. Homopolymers contain one type of building block, or monomer, whereas co-polymers contain more than one type of monomer.

All ranges or numerical values stated herein, whether or not preceded by the term “about” unless stated otherwise are considered to be preceded by the term “about” to account for variations in precision of measurement and functionally equivalent ranges.

As used herein, the terms “comprising,” “comprise” or “comprised,” and variations thereof, are meant to be open ended. The terms “a” and “an” are intended to refer to one or more.

As used herein, the term “patient” or “subject” refers to members of the animal kingdom including but not limited to human beings.

The term “administration” refers to providing or giving a subject a composition, such as a disclosed reverse thermal gel composition by any effective route. Exemplary routes of administration include, but are not limited to, injection (such as subcutaneous, intramuscular, intradermal, intraperitoneal (IP), and intravenous (IV)), oral, sublingual, rectal, transdermal, intranasal, vaginal and inhalation routes.

The term “alkyl” refers to both branched and straight-chain saturated aliphatic hydrocarbon groups. These groups can have a stated number of carbon atoms, expressed as C_{x-y}, where x and y typically are integers. For example, C₅₋₁₀, includes C₅, C₆, C₇, C₈, C₉, and C₁₀. Alkyl groups include, without limitation: methyl, ethyl, propyl, isopropyl, n-, s- and t-butyl, n- and s-pentyl, hexyl, heptyl, octyl, etc. Alkenes comprise one or more double bonds and alkynes comprise one or more triple bonds. These groups include groups that have two or more points of attachment (e.g., alkylene). Cycloalkyl groups are saturated ring groups, such as cyclopropyl, cyclobutyl, or cyclopentyl. Aromatic groups include one or more benzene rings. As used herein, “halo” or “halogen” refers to fluoro, chloro, bromo, and iodo. An amine is a group having the structure —N(R1)(R2). Where R1 and R2 are H, the group is amino.

A polymer “comprises” or is “derived from” a stated monomer if that monomer is incorporated into the polymer. Thus, the incorporated monomer that the polymer comprises is not the same as the monomer prior to incorporation into a polymer, in that at the very least, certain terminal groups are incorporated into the polymer backbone or are removed in the polymerization process. A polymer is said to comprise a specific type of linkage if that linkage is present in the polymer.

The polymers described herein are said to be bioerodible or biodegradable. By that, it is meant that the polymer, once implanted and placed in contact with bodily fluids and tissues, or subjected to other environmental conditions, such as composting, will degrade either partially or completely through chemical reactions, typically and often preferably over a time period of hours, days, weeks or months. Non-limiting examples of such chemical reactions include acid/base reactions, hydrolysis reactions, and enzymatic cleavage. The polymers described herein contain labile ester linkages. The polymer or polymers may be selected so that it degrades over a time period. Non-limiting examples of useful in situ degradation rates include between 12 hours and 5 years, and increments of hours, days, weeks, months or years therebetween. For example, in the context of an drug product to be injected via the intravitreal route, the polymer may preferably degrade over 1, 2, 3, 4, 5, 6, 7, 8, 9, 10, 11, or 12 months, or longer.

An “agent” is any polypeptide, compound, small molecule, organic compound, salt, polynucleotide, or other molecule of interest. In particular examples, an agent is an active agent, such as an antibiotic, analgesic, or procoagulant. In some examples, an agent is a pharmaceutical agent. A “pharmaceutical agent” is a chemical compound, small molecule, or other composition capable of inducing a desired therapeutic or prophylactic effect when properly administered to a subject or a cell.

“Incubating” includes a sufficient amount of time for a drug to interact with a cell.

“Contacting” includes incubating a drug in solid or in liquid form with a cell.

The phrase “effective amount” or “therapeutically effective amount” refers to the amount of agent sufficient to prevent, treat, reduce and/or ameliorate the symptoms and/or underlying causes of any of a disorder or disease. In one embodiment, an “effective amount” is sufficient to reduce or eliminate a symptom of an infection, such as a post-surgery infection. In another embodiment, an effective amount is an amount sufficient to prevent an infection.

The phrase “under conditions sufficient for” is used to describe any environment that permits the desired activity. In one example, under conditions sufficient for includes administering one or more disclosed compositions to a subject to at a concentration sufficient to allow the desired activity. In some examples, the desired activity is reducing or inhibiting a sign or symptom associated with an infection, such as a post-surgical infection. In some examples, the desired activity is promoting wound healing. In some examples, the desired activity is to promote coagulation.

“Biofilm” is a mass of microorganisms attached to a surface, such as a surface of a medical device, and the associated extracellular substances produced by one or more of the attached microorganisms. The extracellular substances are typically polymeric substances that commonly include a matrix of complex polysaccharides, proteinaceous substances and glycopeptides. The microorganisms may include, but are not limited to, bacteria, fungi and protozoa. In a “bacterial biofilm,” the microorganisms include one or more species of bacteria. The nature of a biofilm, such as its structure and composition, may depend on the particular species of bacteria present in the biofilm. Bacteria present in a biofilm are commonly genetically or phenotypically different than corresponding bacteria not in a biofilm, such as isolated bacteria or bacteria in a colony. “Polymicrobic biofilms” are biofilms that include a plurality of bacterial species.

“Inhibiting formation of a biofilm” is a phrase used to describe avoiding the partial or full development or progression of a biofilm, for example, on a surface, such as a surface of an indwelling medical device.

II. Compositions and Methods of Use thereof

Provided is a reverse thermal gel composition comprising a triblock copolymer having the structure B-A-B in which A is one of a polyurethane or poly(ester urethane) group that comprises one or more pendant active groups, blocked active groups or active agents and B is a hydrophilic block that can be PEG of various sizes, hyaluronan of various sizes, poly(vinyl alcohol) or oligo(vinyl alcohol), polycarbohydrage, etc. Examples of poly(ethylene glycol) average molecular weights include 350, 550, 750, 1000, and 1900 Da. The composition is in solution at a lower temperature, e.g., at room temperature and transitions to a gel as the temperature is raised, to form a complete gel at a higher temperature, e.g., physiological (body) temperature (e.g., 35° C.-40° C.). The transition temperature also may be referred to as the Lower Critical Solution Temperature, or LCST) is preferably 30° C. or less or 25° C. to 30° C. As an example, the transition point is above room temperature (RT, for example 25° C.) and physiological temperature (typically 37° C. but there can be individual differences). As a further example, the composition begins transformation as the temperature rises from 25° C. and forms a gel around 33-35° C. and still remains gel at 37° C. In another example, the composition gels between 25° C. to 40° C. and is a liquid solution at temperatures below 25° C. (e.g., 24° C., 23° C., etc.). The triblock copolymer may be converted to a pharmaceutically acceptable salt. In one embodiment, A is a copolymer of a diol (a hydrocarbon comprising aliphatic or aromatic groups and which may be saturated or unsaturated) and a diisocyanate. The diol may be amino-substituted or N-substituted serinol, such as N-boc serinol, in which the N is substituted with one of a hydrogen, a protective group (a removable group that prevents the amine or other desirable moiety from reacting during synthesis of the triblock copolymer), or an active agent. In another embodiment, the N of the N-substituted serinol is —NHR in which R is a protective group, such as carbobenzyloxy; p-methoxybenzyl carbonyl; tert-butyloxycarbonyl; 9-fluorenylmethyloxycarbonyl; benzyl; p-methoxybenzyl; 3,4-dimethoxybenzyl; p-methoxyphenyl; tosyl; nosyl (4-nitrobenzenesulfonyl) and 2-nitrobenzenesulfonyl.

In another embodiment, the diol comprises one or more ester groups, as when it is a reaction product of a cyclic anhydride and a diol comprising one or more pendant active groups, blocked active groups or active agents. For example, the diol in one particular embodiment is the reaction product of succinic anhydride and an N-substituted serinol in which the N is substituted with one of a hydrogen, a protective group, such as carbobenzyloxy; p-methoxybenzyl carbonyl; tert-butyloxycarbonyl; 9-fluorenylmethyloxycarbonyl; benzyl; p-methoxybenzyl; 3,4-dimethoxybenzyl; p-methoxyphenyl; tosyl; nosyl (4-nitrobenzenesulfonyl) and 2-nitrobenzenesulfonyl, or an active agent. In one embodiment, the diol comprises a pendant amino group or an amine. One example of a diisocyanate is hexamethylene diisocyanate (1,6-diisocyanatohexane).

According to one embodiment, the reverse thermal gel composition comprises a copolymer comprising the structure:

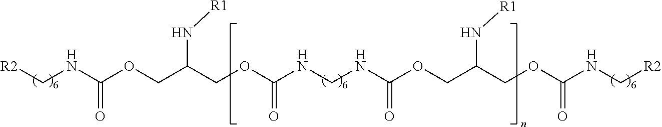

in which R1 is H or a protective group, R2 is isocyanate or —NC(O)-PEG and n is greater than 5, for example and without limitation, 8-30, 8-25 or 18-30. According to another embodiment, the composition comprises a copolymer comprising the structure:

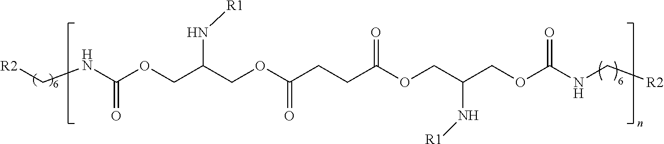

in which R1 is H or a protective group or an active agent, R2 is isocyanate or —NC(O)-PEG and n is greater than 5, for example and without limitation, 8-30, 8-25 or 18-30.

In one embodiment, the triblock copolymer has an average molecular weight of between about 3,000-50,000 Da (Daltons), for instance between 5,000 and 10,000 Da, excluding, when present, the molecular weight of the active agent. The composition may comprise an additional active agent, such as an active agent complexed (non-covalently bound) to a triblock copolymer as described above. According to one non-limiting embodiment, the active agent is the one or more antibiotics, procoagulants, analgesics or a combination thereof.

In some examples, the active agent is at least one, such as at least two, at least three, at least four, at least five, including one, two, three, four, five or more antibiotics, biofilm inhibitors, procoagulant, analgesic, or any combination thereof. In some examples, an active agent is one or more biofilm inhibitors either alone in combination with an antibiotic, procoagulant and/or analgesic. A biofilm inhibitor is any substance which functions to inhibit the formation of a biofilm. For example, a biofilm inhibitor can be a substance which inhibits protein adsorption or biofilm adhesion. In some examples, antibiotics, biocides and/or ion coatings can be used to prevent, reduce and/or inhibit biofilm formation by interfering with the attachment and expansion of immature biofilms. In some examples, a biofilm inhibitor is substance which inhibits protein adsorption or biofilm adhesion and is not an antibiotic. In some examples, a biofilm inhibitor is silver and/or silver ions which are used to interfere with the growth and function of bacteria which form biofilms. In some examples, a biofilm inhibitor is a substance which affects the hydrophobicity or hydrophilicity of the contacting surface, which in turn affects the ability of a biofilm to adhere to said surface. In some examples, a biofilm inhibitor is a substance which either maintains a smooth surface or prevents the development of a rough, high-energy surface which is more conducive to biofilm formation and maturation (such as a substance which maintains a surface roughness with an Ra below 2 μm. In some examples, a biofilm inhibitor is an antimicrobial agents which has been modified with long, flexible polymeric chains (such as N-alkylpyridinium bromide, an antimicrobial agent, modified with a poly(4-vinyl-N-hexylpyridine). In some examples, biofilm inhibitors include several backbone compounds and antimicrobial agents in which the positively charged polycationic chains enable the molecule to stretch out and generate bactericidal activity and inhibit biofilm formation. In some examples, a biofilm inhibitor is polyurethane. In some examples, a biofilm inhibitor is pDMAEMA (poly[2-(dimethylamino)ethyl methacrylate] attached to polyethylene. In some examples, an active agent is one or more antibiotics either alone or in combination with a biofilm inhibitor, procoagulant and/or analgesic. In some examples, the at least one antibiotic, is acyclovir, afloxacin, ampicillin, amphotericin B, atovaquone, azithromycin, ciprofloxacin, clarithromycin, clindamycin, clofazimine, dapsone, diclazaril, doxycycline, erythromycin, ethambutol, fluconazole, fluoroquinolones, foscarnet, ganciclovir, gentamicin, iatroconazole, isoniazid, ketoconazole, levofloxacin, lincomycin, miconazole, neomycin, norfloxacin, ofloxacin, paromomycin, penicillin, pentamidine, polymixin B, pyrazinamide, pyrimethamine, rifabutin, rifampin, sparfloxacin, streptomycin, sulfadiazine, tetracycline, tobramycin, trifluorouridine, trimethoprim sulphate, Zn-pyrithione, and silver salts such as chloride, bromide, iodide and periodate. In some examples, the active agent is one or more antimycobacterial agents and/or one or more broad-spectrum tetracycline antibiotic agents. In some examples, the one or more antimycobacterial agents is rifampicin, rifaximin, dapsone, ampicillin, norfloxacin, silver sulfadiazine, tigecycline, cefoperazone, sulfisoxazole, hydrocortisone/acetic acid, gemifloxacin, rifampin/isoniazid, and rifampin/isoniazid/pyrazinamide. In some examples, the antimycobacterial agent is rifampicin. In some examples, the active agent is one or more broad-spectrum tetracycline antibiotics. In some examples, the broad-spectrum tetracycline antibiotic is minocycline and/or doxycycline. In some examples, the broad-spectrum tetracycline antibiotic is minocycline.

In some examples, the active agent comprises at least one procoagulant agent. A procoagulant is any agent that promotes blood coagulation. In some examples, the one or more procoagulants is fibrinogen, prothrombin, Factor Xa, Aminocaproic acid (Amicar®), conjugated estrogen (IV), desmopressin (DDAVP®, Stimate®), fresh frozen plasma (FFP), Factor 7A (Novoseven®), PCC (factor 9 complex), (Profilnine®, Bebulin®), Factor 9 recombinant (BeneFix®) consists of solely recombinant factor 9, Phytonadione (Mephyton®), Protamin, Tranexamic acid (Cyklokapron®, Lysteda®), adsorbent chemicals (such as zeolites), aprotinin, aminocaproic acid, fibrin and thrombin.

In some examples, the active agent is at least one analgesic. In some examples, the composition comprises at least one of Acetaminophen, Morphine, Ibuprofen, Aspirin, Oxycodone, Tramadol, Codeine, Hydrocodone, Fentanyl, Methadone, Hydrocodone/Acetaminophen, Buprenorphine, Metamizole, Hydromorphone, Gabapentin, Co-codamol, Meperidine, Dextropropoxyphene, Nimesulide, Ketoprofen, Menthol, Butorphanol, Dihydrocodeine, Endorphins, Phenacetin, Co-dydramol, Hydroxyzine, Phenazopyridine, Sufentanil, Xylazine, Alfentanil, Levomepromazine, Phenazone, Hydrocodone/ibuprofen, Amobarbital, Salicylamide, Migraleve, Ziconotide, Methoxyflurane, Magnesium salicylate, RUB A535, Aspergum, Gabapentin enacarbil, Dipipanone, Epibatidine, Thebacon, Dichloralphenazone, Opiorphin, and/or Ohmefentanyl.

Also provided is a method of delivering an active agent to a patient, comprising delivering to the patient a reverse thermal gel composition comprising an active agent and a triblock copolymer having the structure B-A-B in which A is one of a polyurethane or poly(ester urethane) group that comprises one or more pendant active groups, blocked active groups or active agents and B is a poly(ethylene glycol) and which is a gel at 37° C. and a liquid at a temperature below 30° C. In one embodiment, the active agent is at least one antibiotic, biofilm inhibitor, procoagulant, or analgesic agent. In one embodiment, a combined dosage form is provided comprising two or more, such as three, four or more of one or more an antibiotic agent, biofilm inhibitor, analgesic and/or procoagulant or combinations thereof. For example, an antibiotic agent is co-administered with a procoagulant, administrated may be designed so that such substances are asynchronously eluted.

In one embodiment, the composition may be any composition described above, for example a composition comprising a triblock copolymer chosen from one of:

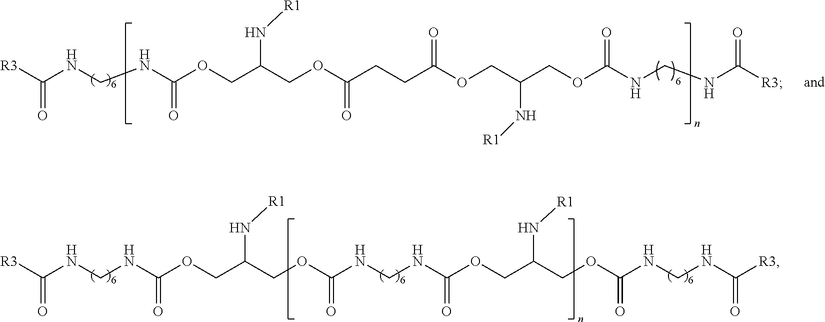

in which R1 is H and R3 is PEG, and one or more antibiotics, biofilm inhibitors, procoagulants, analgesics or a combination thereof so that the composition asynchronously elute two or more types of substances, including antibiotics, biofilm inhibitors, procoagulants, and/or analgesics.

In an embodiment, a method of treating a wound or defect in a patient is provided, comprising delivering to a site in or on the patient a reverse thermal gel composition as described herein including at least one or more antibiotics, biofilm inhibitors, procoagulants, and/or analgesics. In some examples, methods of preventing and/or inhibiting wound bleeding and/or infection are disclosed comprising delivering to a site in or on the patient a reverse thermal gel composition as described herein including at least one or more antibiotics, biofilm inhibitors, procoagulants, and/or analgesics. In some examples, one or more disclosed compositions is administered to prevent and/or inhibit wound bleeding and/or infection following surgery are disclosed. In some examples, methods of preventing and/or inhibiting wound bleeding and/or infection in wounds which by design enclose a foreign body, such as pacemaker implantation pockets, joint replacement sites or neurosurgical sites are disclosed comprising delivering to a site where the foreign body is enclosed a reverse thermal gel composition as described herein including at least one or more, such as at least two antibiotics, biofilm inhibitors, procoagulants, and/or analgesics. In some examples, methods of preventing and/or inhibiting wound bleeding and/or infection in wounds which do not include a foreign body are disclosed, these methods comprising delivering to a site where the wound or bleeding is present a reverse thermal gel composition as described herein including at least one or more antibiotics, biofilm inhibitors, procoagulants, and/or analgesics.

The disclosed compositions can be delivered to the site of interest by methods known to those of skill in the art. For example, where the site in the patient is internal, the composition can be delivered by a needle, cannula, catheter, trochar or any similar devices. The active agents within the polymer composition may vary as depending upon the type of wound and/or infection, the desired duration of treatment or the combination thereof. In some examples, a disclosed composition comprising one or more antibiotics, biofilm inhibitors, procoagulant, and analgesic is administered, such as by injection, to a wound following surgery, such as to a primary cartilaginous joint to prevent or inhibit bleeding or infection. In one particular example, a disclosed composition comprising one or more, such as at least two antibiotics, biofilm inhibitors, procoagulants, and/or analgesics is administered, such as by injection, to a knee following surgery to prevent or inhibit bleeding or infection.

In some examples, a disclosed composition comprising one or more biofilm inhibitors, procoagulants and analgesic is administered, such as by injection, following primary total joint arthroplasty (TJA). In some examples, a disclosed composition comprising one or more antibiotics, biofilm inhibitors, procoagulant, and analgesic is administered, such as by injection, to a subject who is at risk of infection or excessive bleeding, such as a subject who has undergone surgery.

According to one embodiment, a method of making a triblock copolymer is provided. The method comprises: reacting a diol with a diisocyanate to produce a diol product; and PEGylating the diol product. In one embodiment, the idol is synthesized by reacting a diol precursor with a cyclic anhydride. An example of a diol precursor is N-serinol in which the N is substituted with a protective group, such as Boc such that the diol precursor is N-boc-serinol. In another embodiment, the cyclic anhydride is succinic anhydride. Any embodiment of these methods may further comprise complexing the triblock copolymer with an active agent. In one embodiment, the diol precursor is N-serinol, in which the N is substituted with a protective group, for instance N-boc serinol. In yet another embodiment, the diisocyanate is hexamathylene diisocyanate.

The polymer compositions may be modified to include biologically active groups or active agents either covalently bound (attached) to the polymer structure or bound to the structure non-covalently. Active agents can be admixed with the polymer composition, absorbed or adsorbed into the composition. Active agents that may be incorporated into the compositions described herein include, without limitation, anti-inflammatories, such as, without limitation, NSAIDs (non-steroidal anti-inflammatory drugs) such as salicylic acid, indomethacin, sodium indomethacin trihydrate, salicylamide, naproxen, colchicine, fenoprofen, sulindac, diflunisal, diclofenac, indoprofen sodium salicylamide, antiinflammatory cytokines, and antiinflammatory proteins or steroidal anti-inflammatory agents); antibiotics; biofilm inhibitors; anticlotting factors such as heparin, Pebac, enoxaprin, aspirin, hirudin, plavix, bivalirudin, prasugrel, idraparinux, warfarin, coumadin, clopidogrel, PPACK, GGACK, tissue plasminogen activator, urokinase, and streptokinase; growth factors. Other active agents include, without limitation: (1) immunosuppressants; glucocorticoids such as hydrocortisone, betamethisone, dexamethasone, flumethasone, isoflupredone, methylpred-nisolone, prednisone, prednisolone, and triamcinolone acetonide; (2) antiangiogenics such as fluorouracil, paclitaxel, doxorubicin, cisplatin, methotrexate, cyclophosphamide, etoposide, pegaptanib, lucentis, tryptophanyl-tRNA synthetase, retaane, CA4P, AdPEDF, VEGF-TRAP-EYE, AG-103958, Avastin, JSM6427, TG100801, ATG3, OT-551, endostatin, thalidomide, becacizumab, neovastat; (3) antiproliferatives such as sirolimus, paclitaxel, perillyl alcohol, farnesyl transferase inhibitors, FPTIII, L744, antiproliferative factor, Van 10/4, doxorubicin, 5-FU, Daunomycin, Mitomycin, dexamethasone, azathioprine, chlorambucil, cyclophosphamide, methotrexate, mofetil, vasoactive intestinal polypeptide, and PACAP; (4) antibodies; drugs acting on immunophilins, such as cyclosporine, zotarolimus, everolimus, tacrolimus and sirolimus (rapamycin), interferons, TNF binding proteins; (5) taxanes, such as paclitaxel and docetaxel; statins, such as atorvastatin, lovastatin, simvastatin, pravastatin, fluvastatin and rosuvastatin; (6) nitric oxide donors or precursors, such as, without limitation, Angeli's Salt, L-Arginine, Free Base, Diethylamine NONOate, Diethylamine NONOate/AM, Glyco-SNAP-1, Glyco-SNAP-2, (.+−.)-S-Nitroso-N-acetylpenicillamine, S-Nitrosoglutathione, NOC-5, NOC-7, NOC-9, NOC-12, NOC-18, NOR-1, NOR-3, SIN-1, Hydrochloride, Sodium Nitroprusside, Dihydrate, Spermine NONOate, Streptozotocin; and (7) antibiotics, such as, without limitation: acyclovir, afloxacin, ampicillin, amphotericin B, atovaquone, azithromycin, ciprofloxacin, clarithromycin, clindamycin, clofazimine, dapsone, diclazaril, doxycycline, erythromycin, ethambutol, fluconazole, fluoroquinolones, foscarnet, ganciclovir, gentamicin, iatroconazole, isoniazid, ketoconazole, levofloxacin, lincomycin, miconazole, neomycin, norfloxacin, ofloxacin, paromomycin, penicillin, pentamidine, polymixin B, pyrazinamide, pyrimethamine, rifabutin, rifampin, sparfloxacin, streptomycin, sulfadiazine, tetracycline, tobramycin, trifluorouridine, trimethoprim sulphate, Zn-pyrithione, and silver salts such as chloride, bromide, iodide and periodate.

In some examples, the active agent is one or more antimycobacterial agents and/or one or more broad-spectrum tetracycline antibiotic agents. In some examples, the one or more antimycobacterial agents is rifampicin, rifaximin, dapsone, ampicillin, norfloxacin, silver sulfadiazine, tigecycline, cefoperazone, sulfisoxazole, hydrocortisone/acetic acid, gemifloxacin, rifampin/isoniazid, and rifampin/isoniazid/pyrazinamide. In some examples, the antimycobacterial agent is rifampicin. In some examples, the active agent is one or more broad-spectrum tetracycline antibiotics. In some examples, the broad-spectrum tetracycline antibiotic is minocycline and/or doxycycline. In some examples, the broad-spectrum tetracycline antibiotic is minocycline.

In some examples, the active agent comprises at least one, such as one, two, three, four or more biofilm inhibitors.

In some examples, the active agent comprises at least one, such as one, two, three, four or more procoagulant agents. A procoagulant is any agent that promotes blood coagulation. In some examples, the one or more procoagulants is fibrinogen, prothrombin, Factor Xa, Aminocaproic acid (Amicar®), conjugated estrogen (IV), desmopressin (DDAVP®, Stimate®), fresh frozen plasma (FFP), Factor 7A (Novoseven®), PCC (factor 9 complex), (Profilnine®, Bebulin®), Factor 9 recombinant (BeneFix®) consists of solely recombinant factor 9, Phytonadione (Mephyton®), Protamin, Tranexamic acid (Cyklokapron®, Lysteda®), adsorbent chemicals (such as zeolites), aprotinin, aminocaproic acid, fibrin and thrombin.

In some examples, the active agent is at least one, such as one, two, three, four or more analgesics. In some examples, the composition comprises at least one of Acetaminophen, Morphine, Ibuprofen, Aspirin, Oxycodone, Tramadol, Codeine, Hydrocodone, Fentanyl, Methadone, Hydrocodone/Acetaminophen, Buprenorphine, Metamizole, Hydromorphone, Gabapentin, Co-codamol, Meperidine, Dextropropoxyphene, Nimesulide, Ketoprofen, Menthol, Butorphanol, Dihydrocodeine, Endorphins, Phenacetin, Co-dydramol, Hydroxyzine, Phenazopyridine, Sufentanil, Xylazine, Alfentanil, Levomepromazine, Phenazone, Hydrocodone/ibuprofen, Amobarbital, Salicylamide, Migraleve, Ziconotide, Methoxyflurane, Magnesium salicylate, RUB A535, Aspergum, Gabapentin enacarbil, Dipipanone, Epibatidine, Thebacon, Compound analgesic, Dichloralphenazone, Opiorphin, and/or Ohmefentanyl.

In any case, as used herein, any active agent used for prevention or treatment of a condition, such as, for example, a wound or bleeding associated with surgery, is administered in an amount effective to treat or prevent that condition, namely in an amount and in a dosage regimen effective to prevent or reduce the duration and/or severity of the condition.

Active agents that may be bound to the polymer composition include peptides (e.g., ECM epitopes) for functionalizing the gel with a biologically functional group. Useful peptides include or consist of the following amino acid sequences: IKLLI (SEQ ID NO: 1)(anti-apoptotic), REDV (SEQ ID NO: 2), LDV, RGDSP (SEQ ID NO: 3), RGDV (SEQ ID NO: 4), LRGDN (SEQ ID NO: 5), RGDT (SEQ ID NO: 6), YIGSR (SEQ ID NO: 7), TTSWSQ (SEQ ID NO: 8), AEIDGIEL (SEQ ID NO: 9), WYRGRL (SEQ ID NO: 10), SIKVAVS (SEQ ID NO: 11), PDSGR (SEQ ID NO: 12), RNIAEIIKDI (SEQ ID NO: 13), DGEA (SEQ ID NO: 14), VTXG (SEQ ID NO: 15), PRRARV (SEQ ID NO: 16), YEKPGSPPREVVPRPRPGV (SEQ ID NO: 17), RPSLAKKQRFRHRNRKGYRSQRGHSRGR (SEQ ID NO: 18), RIQNLLKITNLRIKFVK (SEQ ID NO: 19), RGD, IKVAV (SEQ ID NO: 20) and IKVAVS (SEQ ID NO: 21). In one example, these oligopeptides are linked via their amine groups to the polymeric structures described herein. In another embodiment, biomolecules are attached or bound to the polymer composition which aid in evasion of an immune response. Non-limiting examples of such peptides are: betaine, derivatives of betaine, and other zwitterionic groups including certain amino acids and their derivatives.

The active agent or any compound or composition may be bound to the polymer in any useful manner, for instance: covalently (including by coordination and by use of a suitable linkers and linking methods as are broadly known and are broadly available in the art, for example linkers and methods of use of linkers are commercially available from Thermo Fisher Scientific, Pierce Protein Research Products, Rockford, Illinois, see also Thermo Scientific Pierce Crosslinking Technical Handbook, 2009 Thermo Fisher Scientific Inc.), by affinity or charge (that is, non-covalently), or by intermixing with the polymer when the composition is in solution phase. Binding of the active agent or any compound or composition by affinity or charge, e.g., by polar, hydrogen bonding, charge (ionic/electrostatic), or van der Waals interactions, may be preferred in many instances because the compound is not free to diffuse prior to or after gelation, as in the case of the active agent being intermixed with the polymer in the composition, or is not covalently modified, which can hamper efficacy of the active agent.

In one embodiment, the active agent is used for prevention or treatment of an ocular disease (disorder or condition), such as a maculopathy, a retinopathy, glaucoma, an inflammatory condition, a bacterial infection, a viral infection or a wound. The composition comprising the active agent is delivered to the eye in any useful fashion. In order to ensure consistent delivery, in one embodiment, the composition is delivered by intravitreal injection. In that case, the composition slowly breaks down in the vitreous humor and the drug is released as the composition breaks down. Suitable active agents include without limitation: antibiotics, anti-inflammatory agents, analgesics, antiangiogenic agents, and growth factors.

Non-limiting examples of antiangiogenic agents include: Macugen (pegaptanib sodium); Lucentis; Tryptophanyl-tRNA synthetase (TrpRS); AdPEDF; VEGF TRAP-EYE; AG-013958; Avastin (bevacizumab); JSM6427; TG100801; ATG3; Perceiva (originally sirolimus or rapamycin); E10030, ARC1905 and colociximab (Ophthotech) and Endostatin. Ranibizumab is currently the standard in the United States for treatment of neovascular AMD. It binds and inhibits all isoforms of VEGF. Although effective in many cases, treatment with ranibizumab requires sustained treatment regimens and frequent intravitreal injections. VEGF Trap is a receptor decoy that targets VEGF with higher affinity than ranibizumab and other currently available anti-VEGF agents. Blocking of VEGF effects by inhibition of the tyrosine kinase cascade downstream from the VEGF receptor also shows promise, and includes such therapies as vatalanib, TG100801, pazopanib, AG013958 and AL39324. Small interfering RNA technology-based therapies have been designed to downregulate the production of VEGF (bevasiranib) or VEGF receptors (AGN211745). Other potential therapies include pigment epithelium-derived factor-based therapies, nicotinic acetylcholine receptor antagonists, integrin antagonists and sirolimus. (See, e.g., Chappelow, A V, et al. Neovascular age-related macular degeneration: potential therapies, Drugs. 2008; 68(8): 1029-36 and Barakat M R, et al. VEGF inhibitors for the treatment of neovascular age-related macular degeneration, Expert Opin Investig Drugs. 2009 May; 18(5): 637-46.

An anti-inflammatory agent may be administered in an amount effective to decrease ocular inflammation and pain associated with a given condition. Steroidal anti-inflammatories are useful, but not preferred because they can cause corneal thinning. Non-steroidal anti-inflammatories (NSAIDs) suitable for ocular use are preferred and include, without limitation: nepafenac (for example and without limitation, Nevenac 0.1%, nepafenac ophthalmic suspension, Alcon Laboratories, Inc.), ketorolac tromethamine (for example and without limitation, Acular LS 0.4%, ketorolac tromethamine ophthalmic suspension, Allergan, Inc.), acetaminophen and bromfenac (for example and without limitation, Xibrom 0.09%, bromfenac ophthalmic suspension, Ista Pharmaceuticals). Thus, also provided herein is a composition comprising the described block copolymer and a pharmaceutically acceptable anti-inflammatory suitable for optical use. These anti-inflammatory compounds often exhibit analgesic effects. In any case, according to the methods described herein, the binding reagent and the anti-inflammatory may be contained in the same composition, but also may be administered separately in a manner effective to treat the infection.

An antibiotic also may be administered along with the block copolymer and, optionally, the anti-inflammatory agent may also be co-administered with the antibiotic, all in an amount effective to treat and/or prevent infection and/or its symptoms. Non-limiting examples of suitable antibiotics include: ciprofloxacin, norfloxacin, afloxacin, levofloxacin, gentamicin, tobramycin, neomycin, erythromycin, trimethoprim sulphate, and polymixin B. Antiviral compounds also may be administered in this manner, such as ganciclovir or fomivirsen.

In any case, as used herein, any active agent used for prevention or treatment of a condition, such as, for example, a maculopathy, such as age-related macular degeneration, diabetic retinopathy, or ocular infection, is administered in an amount effective to treat or prevent that condition, namely in an amount and in a dosage regimen effective to prevent or reduce the duration and/or severity of the condition. As an example, between 1 and 500 mg, for example from 1.25 and 60 mg of AVASTIN (bevacizumab) can be administered in one intravitreal injection when mixed with the block copolymer. The actual amount of active agent present in the composition will depend on the degradation rate of the copolymer and dissociation rate of the agent from the composition. The ordinary intraocular injection dose for AVASTIN is 1.25-2.5 mg per month. If the gel degrades over 6 months, the amount in each “gel” dose would contain 7.5-15 mg AVASTIN, 15-30mg if the composition degrades over a year. Different concentrations and specific activities of active agents will achieve similar results. The composition (drug product) may be administered once or more than once, depending on the duration of the erosion of the block copolymer. For example, the composition can be administered monthly, bimonthly, quarterly or yearly. The amount (e.g., number of drops of drug product) of the drug product administered to the patient, also may vary though the amount administered should not be either harmful to the patient or interfere other than insubstantially with functioning, such as vision.

Non-limiting examples of growth factors suitable for ocular use include: non-mitogenic human acidic fibroblast growth factor (nm-haFGF), neurotrophin nerve growth factor (NGF), epidermal growth factors (EGF), brain-derived neurotrophic factor (BDNF), glial cell line-derived neurotrophic factor (GDNF), neurotrophin-3 and eye-derived growth factor(s) (EDGF).

In one embodiment, a combined dosage form is provided comprising two or more of an anti-angiogenic agent, an anti-inflammatory agent, an antibiotic agent and a growth factor. For example, either an antibiotic or antiviral agent may be co-administered with an anti-inflammatory agent.

In one embodiment, a combined dosage form is provided comprising two or more of an antibiotic agent, a procoagulant agent, and an analgesic. For example, either an antibiotic agent or procoagulant agent may be co-administered with an analgesic agent. In some examples, a composition comprises at least three active agents comprising three or more of an antibiotic agent, a procoagulant agent, and an analgesic.

In any use for the prevention and/or treatment of any condition in a patient, a person of ordinary skill in the pharmaceutical and medical arts will appreciate that it will be a matter of simple design choice and optimization to identify a suitable dosage regimen for treatment of any given condition using the delivery systems/compositions described herein. As such, the composition may comprise a carrier, such as an opthamologically-acceptable carrier, which comprises acceptable excipients, such as, without limitation, one or more suitable: vehicle(s), solvent(s), diluent(s), pH modifier(s), buffer(s), salt(s), colorant(s), rheology modifier(s), lubricant(s), antifoaming agent(s), hydrogel(s), surfactant(s), emulsifier(s), adjuvant(s), preservative(s), phospholipid(s), fatty acid(s), mono-, di- and tri-glyceride(s) and derivatives thereof, wax(es), oil(s) and water, as are broadly known in the pharmaceutical arts.

The compositions described herein may find use as cell growth scaffolds. Cells may be microintegrated within a cell growth matrix using a variety of methods. In likely the simplest embodiment to implement, the cells are mixed with the copolymer when it is a miscible liquid, below the gelation temperature. The following are examples of methods used to incorporate cells into traditional cell scaffolds that are gelled or solid at the time of cell incorporation. They may be useful in case where a cell type would need to be preconditioned to the matrix prior to implantation. In the context of the present disclosure, the gel may be warmed until it gels and then cells are incorporated, for example, as follows. In each case, the gel would need to be kept above the gelation temperature throughout. However, reduction of the temperature until the gel/cell mixture is a miscible liquid may be desirable for the purpose of either facilitating delivery to a patient through a needle or catheter, or for isolating cells, in that the solution can be centrifuged to pellet the cells.

In one example, a gel is submersed in an appropriate growth medium for the cells to be incorporated, and then directly exposed to the cells. The cells are allowed to proliferate on the surface and interstices of the matrix. The matrix is then removed from the growth medium, washed if necessary, and implanted. Cells of interest also can be dissolved into an appropriate solution (e.g., a growth medium or buffer) and then sprayed onto a growth matrix. This method is particularly suitable when a highly cellularized tissue engineered construct is desired. In one embodiment, pressure spraying (i.e., spraying cells from a nozzle under pressure) is used to deposit the cells. In another, the cells are electrosprayed onto the non-woven mesh during electrodeposition. Electrospraying involves subjecting a cell-containing solution with an appropriate viscosity and concentration to an electric field sufficient to produce a spray of small charged droplets of solution that contain cells.

Many cell types require a support cell population or matrix in order to, for example, survive, grow, propagate or differentiate. As indicated above, cells can be mixed with the composition at a temperature below the gelation temperature for the composition. Next, the temperature of the composition is raised to produce a gel containing the cells. The cells are grown at a temperature at which the composition is gelled. Lastly, the cells can be removed from the gel by first lowering the temperature of the composition to below the gelation temperature to “melt” the gel, and then the cells are washed, e.g., with medium, saline or PBS (Phosphate-Buffered Saline) to remove the polymer composition. By this method specific shapes of tissue may be generated, for instance by growing the cells in a mold, and letting the cells grow/differentiate until cell-cell interaction is achieved. Once the cells or tissue is grown, the cells or tissue can then be washed free of any remaining polymer.

The cells that may be incorporated on or into the gel includes stem cells such as adipose or neural stem cells; progenitor (precursor) cells; smooth muscle cells; skeletal myoblasts; myocardial cells; endothelial cells; endothelial progenitor cells; bone-marrow derived mesenchymal cells and genetically modified cells. In certain embodiments, the genetically modified cells are capable of expressing a therapeutic substance, such as a growth factor. Examples of suitable growth factors include angiogenic or neurotrophic factor, which optionally may be obtained using recombinant techniques. Non-limiting examples of growth factors include basic fibroblast growth factor (bFGF or FGF-2), acidic fibroblast growth factor (aFGF), nerve growth factor (NGF), vascular endothelial growth factor (VEGF), hepatocyte growth factor (HGF), insulin-like growth factors (IGF), transforming growth factor-beta pleiotrophin protein, midkine protein.

In one embodiment of the methods described herein, the composition, with or without cells, growth factors, active agents (such as one or more antibiotics, biofilm inhibitors, procoagulants and/or analgesics), etc. is injected or deposited at the site of a wound to heal the wound. Wrapping or covering the wound, e.g., in bandages can insure that the composition remains at a temperature at which it is a gel. The composition with or without cells, growth factors, active agents (such as one or more antibiotics, biofilm inhibitors, procoagulants and/or analgesics), etc., can be injected or otherwise administered at any point in or on a patient. For instance a catheter, cannula, trochar, syringe, etc. can be used to deliver the composition to a desired location. In one embodiment, a method of growing nerve cells, such as a method of repairing a nerve, is provided. In one embodiment, the composition is implanted in a wound to a nerve so that nerve cells can populate the hydrogel and nerve structure and function is regenerated. For example, the composition can be implanted at a site of a spinal cord wound (see below for an example) and the nerve tissue is regenerated.

Pharmaceutically acceptable salts are, because their solubility in water is greater than that of the initial or basic compounds, particularly suitable for medical applications. These salts have a pharmaceutically acceptable anion or cation. Suitable pharmaceutically acceptable acid addition salts of the compounds of the invention include, without limitation, salts of inorganic acids such as hydrochloric acid, hydrobromic, phosphoric, metaphosphoric, nitric and sulfuric acid, and of organic acids such as, for example, acetic acid, benzenesulfonic, benzoic, citric, ethanesulfonic, fumaric, gluconic, glycolic, isethionic, lactic, lactobionic, maleic, malic, methanesulfonic, succinic, p-toluenesulfonic and tartaric acid. Suitable pharmaceutically acceptable basic salts include without limitation, ammonium salts, alkali metal salts (such as sodium and potassium salts), alkaline earth metal salts (such as magnesium and calcium salts), and salts of trometamol (2-amino-2-hydroxymethyl-1,3-propanediol), diethanolamine, lysine or ethylenediamine. Pharmaceutically acceptable salts may be prepared from parent compounds by any useful method, as are well known in the chemistry and pharmaceutical arts.

As described above, the compositions described herein are useful for drug delivery, especially were systemic treatment is not necessary or dangerous. One or more therapeutic agents may be included in the compositions and the composition is delivered to a site in a patient, where the composition gels. Delivery of the composition is limited by the rate of degradation of the polymeric component of the composition. As such, the composition may be useful in treating tumors, for example, by complexing an anticancer agent with the polymeric component of the composition and delivering the composition to the site of a tumor, where it slowly releases the anticancer agent. Likewise, these compositions may find use in treating localized conditions, such as abscesses. The composition may be useful in delivering steroids at a constant rate, for example in the case of testosterone, where less than optimal injections, topical gels and patches are the norm, or contraceptives.

### EXAMPLE

### Inhibition of Infection Following Pacemaker Implantation

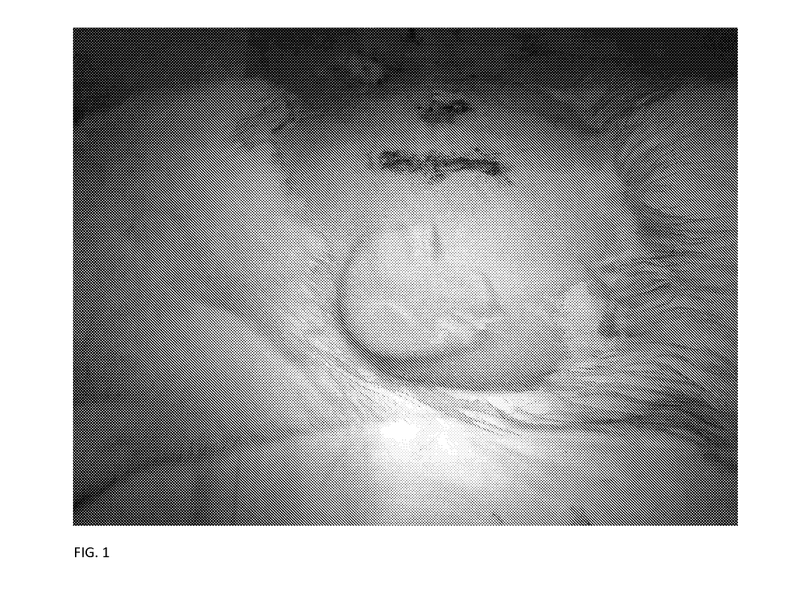

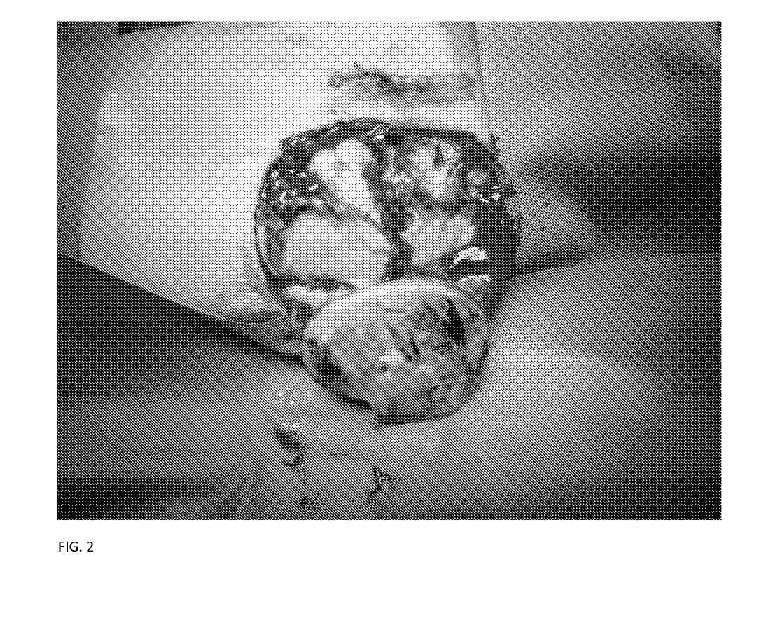

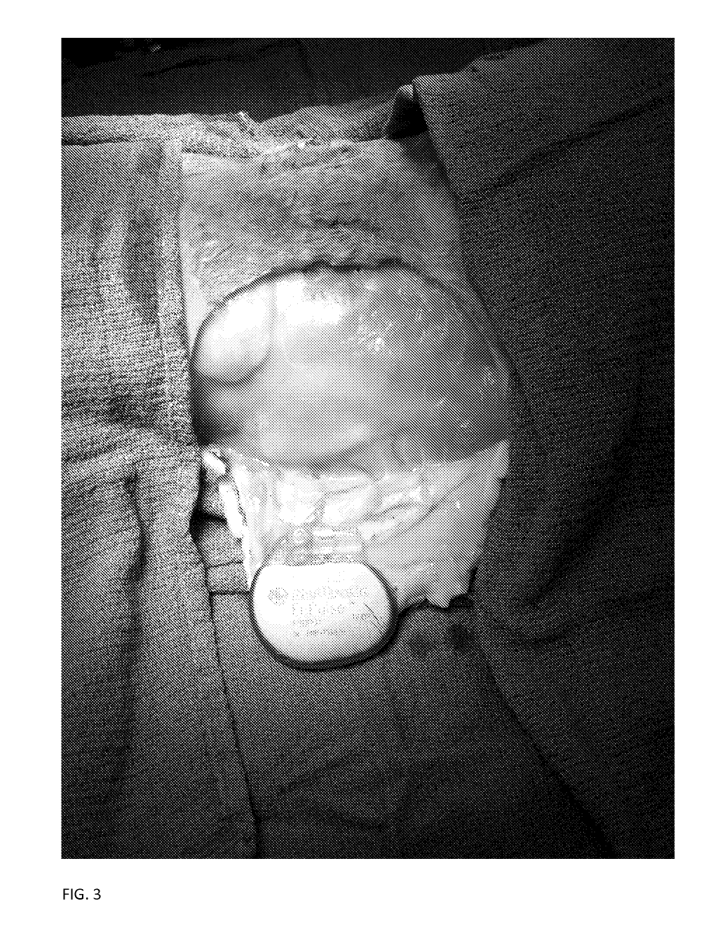

Rabbits (New Zealand White, 5-6 months old, male, 4-5 kg) were utilized for these studies. In each rabbit, a paraspinal subcutaneous pocket was formed using blunt dissection. Into every pocket was placed a commercial pacemaker pulse generator and 1 cc of bacterial inoculum containing 10⁷ colony-forming units of methicillin-resistant staphylococcus aureus (MRSA; ATCC catalog number 43300). In 8 pockets, the pacemaker was placed into an AIGISRx® pouch prior to placing it into the pocket. In 8 other pockets, 2 cc of Theragel was injected just prior to wound closure. At 1 week, pockets were opened under sterile technique and contents plated onto commercial Petri dishes, using standard laboratory protocol procedures. Dishes were incubated for 72 hours using. Any growth was identified using standard bacteriologic techniques. MRSA was isolated from all 8 pockets in which neither AIGISRx® nor Theragel were included. No bacteria were isolated from any of the pockets in which either of these products were included (see FIGS. 1-3). FIG. 1 is a graph illustrating cumulative release of minocycline HCl and rifampicin.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A composition, comprising:
a reverse thermal gel composition comprising a triblock copolymer, or a pharmaceutically acceptable salt thereof, having the structure B-A-B in which A is one of a polyurethane or poly(ester urethane) group and B is a hydrophilic block, wherein the composition is a gel at 25° C. to 40° C. and a liquid solution at a lower temperature; and
at least two active agents selected from the group consisting of a biofilm inhibitor, an antibiotic agent, a procoagulant agent and an analgesic agent.

2. The composition of claim 1, wherein A is a copolymer of a diol and a diisocyanate.

3. The composition of claim 2, wherein the diol is an amino-substituted or N-substituted serinol in which the N is substituted with one of a hydrogen, a protective group, or an active agent.

4. The composition of claim 3, wherein the N of the N-substituted serinol is —NHR in which R is a protective group.

5. The composition of claim 4, wherein R is selected from the group consisting of carbobenzyloxy; p-methoxybenzyl carbonyl; tert-butyloxycarbonyl; 9-fluorenylmethyloxycarbonyl; benzyl; p-methoxybenzyl; 3,4-dimethoxybenzyl; p-methoxyphenyl; tosyl; nosyl (4-nitrobenzenesulfonyl) and 2-nitrobenzenesulfonyl.

6. The composition of claim 4, wherein R is tert-butyloxycarbonyl.

7. The composition of claim 2, wherein the diol comprises one or more ester groups.

8. The composition of claim 7, wherein the diol is a reaction product of a cyclic anhydride and a diol comprising one or more pendant active groups, blocked active groups or active agents.

9. The composition of claim 8, wherein the diol is the reaction product of succinic anhydride and the diol is an N-substituted serinol in which the N is substituted with one of a hydrogen, a protective group, or an active agent.

10. The composition of claim 2, wherein the diol comprises a pendant amino group or an amine.

11. The composition of claim 2, wherein the diisocyanate is hexamethylene diisocyanate (1,6-diisocyanatohexane).

12. The composition of claim 1, comprising a copolymer comprising the structure: 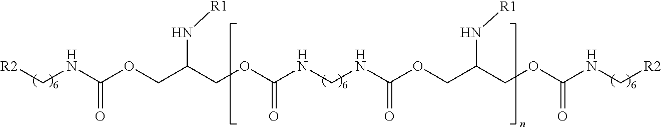
in which R1 is H or a protective group, R2 is isocyanate or —NC(O)-PEG and n is greater than 5, 8-30, 8-25 or 18-30.

13. The composition of claim 1, comprising a copolymer comprising the structure: 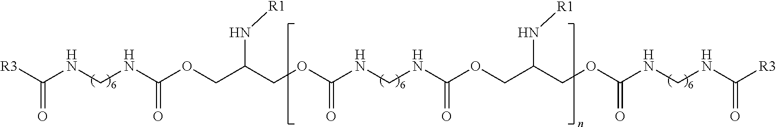
in which R1 is H or a protective group, R3 is PEG and n is greater than 5, 8-30, 8-25 or 18-30.

14. The composition of claim 1, comprising a copolymer comprising the structure: 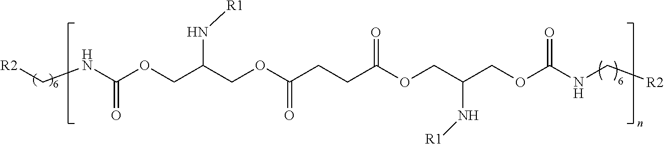
in which R1 is H or a protective group or an active agent, R2 is isocyanate or —NC(O)-PEG and n is greater than 5, 8-30, 8-25 or 18-30.

15. The composition of claim 1, comprising a copolymer comprising the structure: 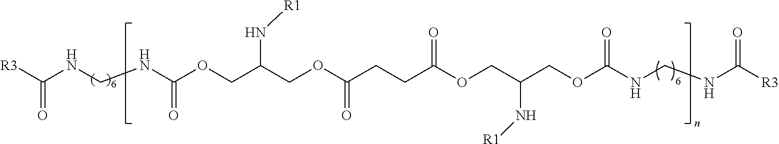
in which R1 is H or a protective group, R3 is PEG and n is greater than 5, 8-30, 8-25 or 18-30.

16. The composition of claim 1, wherein the triblock copolymer having an average molecular weight of between about 5,000 and 10,000 Da (Daltons), excluding the molecular weight of the at least two active agents.

17. The composition of claim 1, wherein A is one of a polyurethane or poly(ester urethane) group that comprises one or more pendant charged or active groups.

18. The composition of claim 17, wherein the one or more pendant charged or active groups is —NH₂.

19. The composition of claim 18, wherein the NH₂ is covalently linked or non-covalently bound to the at least two active agents or biologically functional groups.

20. The composition of claim 1, wherein the at least two active agents are complexed (non-covalently bound) to the triblock copolymer.

21. The composition of claim 1, wherein the antibiotic agent is an antimycobacterial agent and/or broad-spectrum tetracycline antibiotic agent.

22. The composition of claim 21, wherein the antimycobacterial agent is rifampicin, rifaximin, dapsone, ampicillin, norfloxacin, silver sulfadiazine, tigecycline, cefoperazone, sulfisoxazole, hydrocortisone/acetic acid, gemifloxacin, rifampin/isoniazid, rifampin/isoniazid/pyrazinamide.

23. The composition of claim 21, wherein the broad-spectrum tetracycline antibiotic agent is minocycline and/or doxycycline.

24. The composition of claim 1, wherein the procoagulant agent is fibrinogen, prothrombin, factor Xa, and/or thrombin.

25. The composition of claim 1, in which the triblock copolymer is one of: 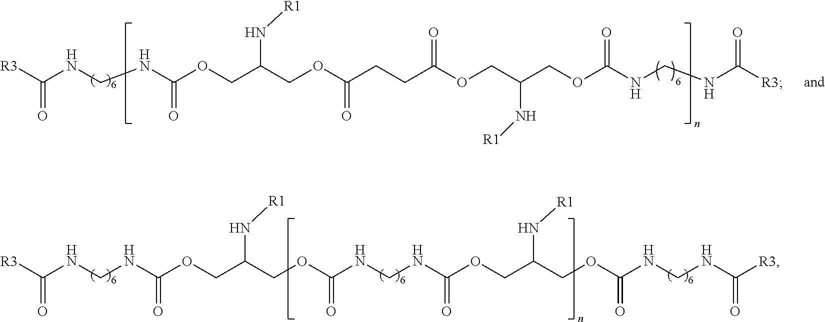
in which R1 is H and R3 is PEG, complexed with an antibiotic agent and a procoagulant agent.

26. The composition of claim 25, wherein the antibiotic agent is rifampicin or minocycline.

27. The composition of claim 1, wherein B is a polyethylene glycol.

28. A method of preventing or inhibiting bleeding or infection in a subject, comprising:
administering an effective concentration of a composition of claim 1 to a site in or on the subject in need thereof, thereby preventing or inhibiting bleeding or infection following a surgical procedure.

29. The method of claim 28, wherein the site in the subject is internal and the composition is delivered by a needle, cannula, catheter, or trochar.

30. The method of claim 28, wherein the composition is delivered to a primary cartilaginous joint.

31. The method of claim 28, wherein the composition is delivered to a subject following knee surgery.

32. The method of claim 28, wherein the composition is delivered to a subject's following primary total joint arthroplasty (TJA).

33. The method of claim 28, wherein the composition is delivered to a site of nerve damage in the patient.

34. The method of claim 33, wherein the site of nerve damage is the patient's spinal cord.


SEQUENCE LISTING (21 sequences)

SEQ ID NO: 1 - protein, 5 aa, Homo sapiens
IKLLI

SEQ ID NO: 2 - protein, 4 aa, Homo sapiens
REDV

SEQ ID NO: 3 - protein, 5 aa, Homo sapiens
RGDSP

SEQ ID NO: 4 - protein, 4 aa, Homo sapiens
RGDV

SEQ ID NO: 5 - protein, 5 aa, Homo sapiens
LRGDN

SEQ ID NO: 6 - protein, 4 aa, Homo sapiens
RGDT

SEQ ID NO: 7 - protein, 5 aa, Homo sapiens
YIGSR

SEQ ID NO: 8 - protein, 6 aa, Homo sapiens
TTSWSQ

SEQ ID NO: 9 - protein, 8 aa, Homo sapiens
AEIDGIEL

SEQ ID NO: 10 - protein, 6 aa, Homo sapiens
WYRGRL

SEQ ID NO: 11 - protein, 7 aa, Homo sapiens
SIKVAVS

SEQ ID NO: 12 - protein, 5 aa, Homo sapiens
PDSGR

SEQ ID NO: 13 - protein, 10 aa, Homo sapiens
RNIAEIIKDI

SEQ ID NO: 14 - protein, 4 aa, Homo sapiens
DGEA

SEQ ID NO: 15 - protein, 4 aa, Homo sapiens
  misc_feature (3)..(3): Xaa can be any naturally occurring amino acid
VTXG

SEQ ID NO: 16 - protein, 6 aa, Homo sapiens
PRRARV

SEQ ID NO: 17 - protein, 19 aa, Homo sapiens
YEKPGSPPREVVPRPRPGV

SEQ ID NO: 18 - protein, 28 aa, Homo sapiens
RPSLAKKQRFRHRNRKGYRSQRGHSRGR

SEQ ID NO: 19 - protein, 17 aa, Homo sapiens
RIQNLLKITNLRIKFVK

SEQ ID NO: 20 - protein, 5 aa, Homo sapiens
IKVAV

SEQ ID NO: 21 - protein, 6 aa, Homo sapiens
IKVAVS